(19) United States
(12) Patent Application Publication
(10) Pub. No.: US 2023/0293244 A1
(43) Pub. Date: Sep. 21, 2023

(54) SYSTEMS AND METHODS FOR HYBRID MOTION PLANNING

(71) Applicant: Mazor Robotics Ltd., Caesarea (IL)

(72) Inventors: Avraham Turgeman, Beer Yaakov (IL); Yaniv Ben Zriham, Binyamina (IL); Ben Yosef Hai Ezair, Haifa (IL); Eliyahu Zehavi, Tel Aviv (IL); Moshe Shoham, Hoshaya (IL)

(21) Appl. No.: 18/169,069

(22) Filed: Feb. 14, 2023

Related U.S. Application Data
(60) Provisional application No. 63/321,472, filed on Mar. 18, 2022.

(51) Int. Cl. A61B 34/20; A61B 34/32; A61B 34/00

(52) U.S. Cl. CPC A61B 34/20; A61B 34/32; A61B 34/25; A61B 2034/2074; A61B 2034/254

(57) ABSTRACT

A system according to at least one embodiment of the present disclosure includes a processor; and a memory storing data thereon that, when processed by the processor, cause the processor to: move a first robotic arm from a first state to a second state; update, based on the moving of the first robotic arm from the first state to the second state, a first status identifier associated with the first robotic arm in a combination state table, the combination state table associated with the first robotic arm and a second robotic arm; determine, based on the first status identifier and the combination state table, a set of permissive states and a set of non-permissive states for the second robotic arm; and prevent the second robotic arm in a third state from performing one or more actions that interfere with the first robotic arm being in the second state.

### CROSS REFERENCE TO RELATED APPLICATIONS

This application claims the benefit of U.S. Provisional Application No. 63/321,472, filed on Mar. 18, 2022, and entitled “Systems and Methods for Hybrid Motion Planning”, which application is incorporated herein by reference in its entirety.

### BACKGROUND

The present disclosure is generally directed to motion planning, and relates more particularly to hybrid motion planning for robotic arms.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A system according to at least one embodiment of the present disclosure comprises: a processor; and a memory storing data thereon that, when processed by the processor, cause the processor to: move a first robotic arm from a first state to a second state; update, based on the moving of the first robotic arm from the first state to the second state, a first status identifier associated with the first robotic arm in a combination state table, the combination state table associated with the first robotic arm and a second robotic arm; determine, based on the first status identifier and the combination state table, a set of permissive states and a set of non-permissive states for the second robotic arm; and prevent the second robotic arm in a third state from performing one or more actions that interfere with the first robotic arm being in the second state.

Any of the aspects herein, wherein the combination state table comprises a matrix of binary values representative of a plurality of states, wherein the plurality of states comprises the first state, the second state, and the third state, wherein the combination state table represents a permissive state as a first known value, and wherein the combination state table represents a non-permissive state as a second known value.

Any of the aspects herein, wherein the set of non-permissive states includes the second state, and wherein the one or more actions includes the second robotic arm moving into the second state.

Any of the aspects herein, wherein the set of permissive states includes a fourth state, and wherein the data further cause the processor to: move the second robotic arm from a third state into a fourth state; update, based on the moving of the second robotic arm from the third state to the fourth state, a second status identifier associated with the second robotic arm in the combination state table; and determine, based on the second status identifier and the combination state table, a permissive move and a non-permissive move for the first robotic arm.

Any of the aspects herein, wherein the data further cause the processor to: prevent the first robotic arm from performing the non-permissive move.

Any of the aspects herein, wherein the combination state table includes a finite number of values.

Any of the aspects herein, wherein the plurality of states comprises a home state, a tool cabinet state, a scanning state, an action state, and an implant state.

Any of the aspects herein, wherein the combination state table includes a finite number of values, and wherein the plurality of states comprises a home state, a tool cabinet state, a scanning state, and action state, and an implant state.

Any of the aspects herein, wherein at least one of the first robotic arm or the second robotic arm comprises at least two degrees-of-freedom.

A method according to at least one embodiment of the present disclosure comprises: causing a first robotic arm to move from a first state to a second state; updating, based on the first robotic arm moving from the first state to the second state, a first status identifier of the first robotic arm in a combination state table, the combination state table associated with both the first robotic arm and a second robotic arm; determining, based on the first status identifier and the combination state table, at least one non-permissive state for the second robotic arm; and preventing the second robotic arm in a third state from moving to the at least one non-permissive state while the first robotic arm is in the second state.

Any of the aspects herein, wherein the combination state table comprises a matrix of binary values representing a plurality of states, wherein the plurality of states comprise the first state, the second state, and the third state, wherein the combination state table represents a permissive state with a first known value, and wherein the combination state table represents a non-permissive state with a second known value.

Any of the aspects herein, wherein the at least one non-permissive state includes the second state.

Any of the aspects herein, further comprising: causing the second robotic arm to move from the third state to a fourth state; updating, based on the moving of the second robotic arm from the third state to the fourth state, a second status identifier associated with the second robotic arm in the combination state table; determining, based on the second status identifier and the combination state table, a permissive move and a non-permissive move for the first robotic arm; and preventing the first robotic arm from performing the non-permissive move.

Any of the aspects herein, further comprising: causing the second robotic arm to move from the third state to a fourth state; updating, based on the moving of the second robotic arm from the third state to the fourth state, a second status identifier associated with the second robotic arm in the combination state table; determining, based on the second status identifier and the combination state table, a permissive move and a non-permissive move for the first robotic arm; and preventing the first robotic arm from performing the non-permissive move, wherein the at least one non-permissive state includes the second state.

Any of the aspects herein, wherein the second robotic arm begins moving from the third state to the fourth state along a first navigation path, and wherein preventing the second robotic arm from moving to the at least one non-permissive state includes: updating, based on the at least one non-permissive state, the first navigation path to a second navigation path, wherein the first navigation path intersects the second state, and wherein the second navigation path avoids intersecting the second state.

Any of the aspects herein, further comprising: permitting, upon the first robotic arm exiting the second state, the second robotic arm to move along the first navigation path.

Any of the aspects herein, further comprising: permitting, upon the first robotic arm exiting the second state, the second robotic arm to move along the first navigation path, wherein the second robotic arm begins moving from the third state to the fourth state along a first navigation path, and wherein preventing the second robotic arm from moving to the at least one non-permissive state includes: updating, based on the at least one non-permissive state, the first navigation path to a second navigation path, wherein the first navigation path intersects the second state, and wherein the second navigation path avoids intersecting the second state.

Any of the aspects herein, wherein the plurality of states comprises a home state, a tool cabinet state, a scanning state, an action state, and an implant state.

Any of the aspects herein, wherein at least one of the first robotic arm or the second robotic arm comprises at least two degrees-of-freedom.

An apparatus according to at least one embodiment of the present disclosure comprises: a first robotic arm disposed in a first state of a plurality of states; a second robotic arm disposed in a second state of the plurality of states; a processor; and a memory storing data thereon that, when processed by the processor, cause the processor to: move, at a first time, the first robotic arm from the first state to a third state of the plurality of states; update, based on the moving of the first robotic arm from the first state to the third state, a first status identifier associated with the first robotic arm in a combination state table, the combination state table associated with both the first robotic arm and the second robotic arm; determine, based on the first status identifier and the combination state table, a set of non-permissive states for the second robotic arm; and prevent, at a second time later than the first time, the second robotic arm from entering a non-permissive state of the set of non-permissive states.

Any of the aspects herein, wherein preventing the second robotic arm from entering a non-permissive state includes: diverting the second robotic arm from a first navigation path to a second navigation path, wherein the first navigation path intersects with the third state, and wherein the second navigation path avoids intersecting the third state.

Any of the aspects herein, wherein the data further cause the processor to: move, at a third time later than the second time, the first robotic arm from the third state to a fourth state of the plurality of states; update, based on the move of the first robotic arm from the third state to the fourth state, the first status identifier associated with the first robotic arm in the combination state table; and permit the second robotic arm to traverse the first navigation path.

Any of the aspects herein, wherein the plurality of states include a home state, a tool cabinet state, a scanning state, an action state, and an implant state.

Any of the aspects herein, wherein preventing the second robotic arm from entering a non-permissive state includes: diverting the second robotic arm from a first navigation path to a second navigation path, wherein the first navigation path intersects with the third state, and wherein the second navigation path avoids intersecting the third state, wherein the data further cause the processor to: move, at a third time later than the second time, the first robotic arm from the third state to a fourth state of the plurality of states; update, based on the move of the first robotic arm from the third state to the fourth state, the first status identifier associated with the first robotic arm in the combination state table; and permit the second robotic arm to traverse the first navigation path, and wherein the plurality of states include a home state, a tool cabinet state, a scanning state, an action state, and an implant state.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Z0).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a block diagram of two robotic arms according to at least one embodiment of the present disclosure;

FIG. 3 is a diagram of a state table according to at least one embodiment of the present disclosure;

FIG. 4A is a diagram of hybrid motion of the two robotic arms according to at least one embodiment of the present disclosure;

FIG. 4B is a diagram of additional hybrid motion of the two robotic arms according to at least one embodiment of the present disclosure; and

FIG. 5 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Using two or more, seven degree-of-freedom (“DOF”) robotic arms in a shared and complex environment, such as a surgical environment, may require robust solutions for coordinating motion of the two robotic arms to prevent collisions. Such collisions may include collisions between the two robotic arms. The working volume may be or comprise a volume containing patient anatomy or other anatomical elements related to a surgery or surgical procedure being performed and into which the two robotic arms may enter while performing or assisting with the surgery or surgical procedure. The working volume may also be occupied by a surgeon or a surgical assistant, creating more congestion in the working volume

According to at least one embodiment of the present disclosure, a hybrid motion planner may be used to control the two or more robotic arms. The hybrid motion planner may include a single arm planner (that is, a planner that plans the motion for a single robotic arm) from a starting configuration to a final configuration. In one embodiment, the single arm planner may determine the starting configuration, the final configuration, and the path therebetween using one or more planning applications. Additionally, the hybrid motion planner may include a state-machine, table, or the like, where each state describes a location or sector in three-dimensional (3D) space. The hybrid motion planner may also include a table of values that indicate free and occupied slots between the arms. The movement of one robotic arm from a first space to a second space may change a status identifier associated with the robotic arm, and the hybrid motion planner may consult the table to determine where the second robotic arm can and cannot move. By verifying states available or unavailable to each robotic arm with quick and easy reference to the table, both robotic arms can be controlled to perform different tasks at the same time without collision. The performance of the different tasks without collision saves precious operation time in the operating room, thereby increasing patient safety and outcomes.

The single arm planner may include or comprise a planning solver that is configured to solve movement solutions for the first robotic arm using various mathematical techniques (e.g., inverse kinematics, pseudo Jacobian inversion, gradient-descent, stochastic gradient descent, backpropagation, other numerical optimization techniques, etc.). Each of the two or more robotic arms working in a shared working volume may have their own independent single arm planner and their own independent planning solver. The single arm planners of each robotic arm may efficiently and safely coordinate motions (or lack thereof) with one another by updating and referencing an appropriate table as disclosed herein.

Embodiments of the present disclosure provide technical solutions to one or more of the problems of (1) managing multiple robotic arms working in a shared volume; (2) managing/avoiding potential collisions between one or more robotic arms and other items (e.g., other robotic arms, patient anatomy, surgeons, members of surgical staff, etc.) in a working volume; and (3) maneuvering two robotic arms simultaneously in an efficient manner where each robotic arm includes its own motion planner/controller.

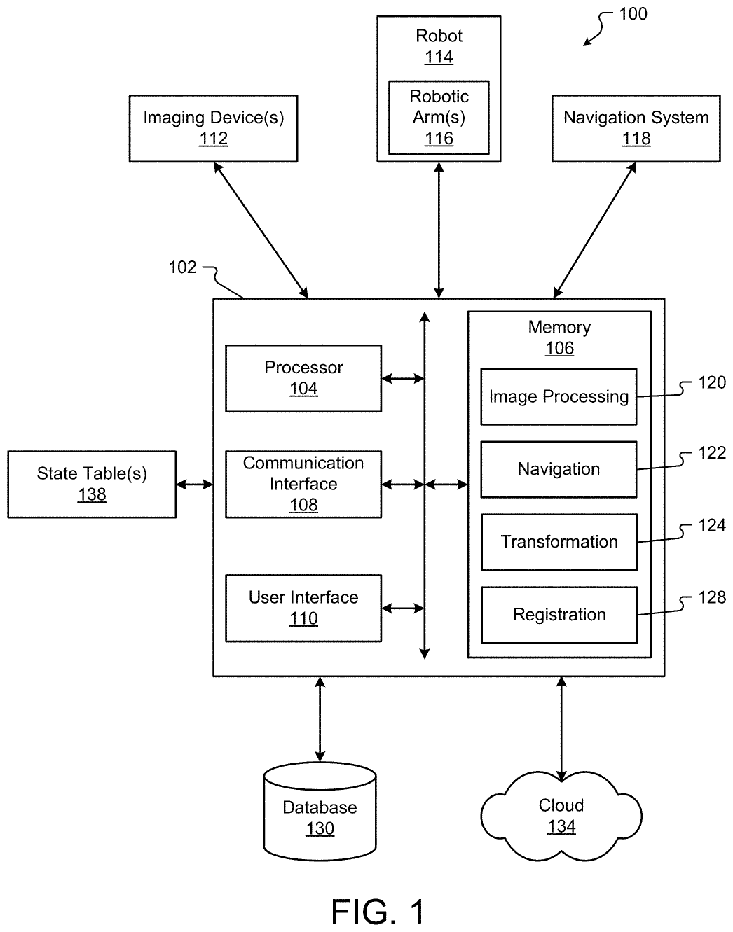

Turning first to FIG. 1, a block diagram of a system 100 according to at least one embodiment of the present disclosure is shown. The system 100 may be used to determine the motion of a first robotic arm; determine a set of allowed states for a second robotic arm to occupy; facilitate a surgery or surgical procedure; monitor the movement of the first and/or second robotic arm; control, pose, and/or otherwise manipulate a surgical mount system, a surgical arm, and/or surgical tools attached thereto; and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 500 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, navigation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the computing device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computing device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the navigation system 118 may comprise multiple independent processors or controllers that each independently control a robotic arm with reference to a shared state table or each with reference to a separate state table. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 may include one or more state tables 138. The state tables 138 may be referenced by one or more controllers or processors in controlling the robotic arms 116. For instance, and as further discussed below, a first controller of the navigation system 118 may control a first robotic arm and a second controller of the navigation system 118 may control a second robotic arm with reference to a state table, such that the two robotic arms avoid collisions with one another. In some embodiments, the state tables 138 may include information about the states of the two robotic arms, such that when one robotic arm is in a first state, the second controller may consult the state tables 138 and, based on the information in the state tables 138, avoid navigating the second robotic arm into the first state.

The system 100 or similar systems may be used, for example, to carry out one or more aspects of the method 500 described herein. The system 100 or similar systems may also be used for other purposes.

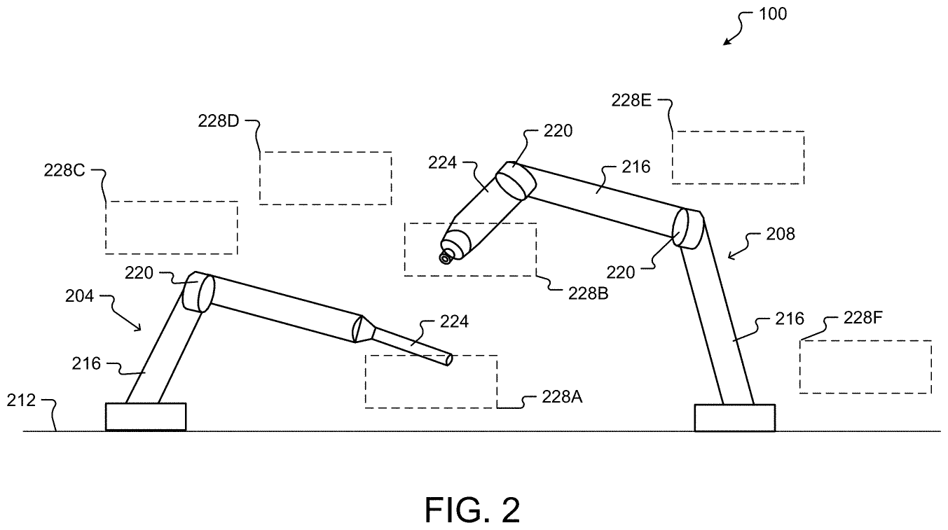

Turning now to FIG. 2, two robotic arms are shown in accordance with at least one embodiment of the present disclosure. While only two robotic arms are depicted, it should be appreciated that the system 100 may include two, three, four, or more robotic arms working in a shared working environment. The illustration of two robotic arms is for ease of discussion and understanding. FIG. 2 illustrates a first robotic arm 204 and a second robotic arm 208 (of which one or both may be similar to or the same as the robotic arm(s) 116). In some embodiments, the first robotic arm 204 and/or the second robotic arm 208 may be mounted or connected to a floor 212, such as a floor of an operating room. In other embodiments, the first robotic arm 204 and/or the second robotic arm 208 may be connected, mounted to, or mechanically coupled with other components or locations in the operating room, such as a patient bed, a wall or a ceiling of the operating room, combinations thereof, etc. The first robotic arm 204 and/or the second robotic arm 208 may comprise one or more segments 216 connected by one or more joints 220. The additional segments 216 and joints 220 may enable in the first robotic arm 204 and/or the second robotic arm 208 to have any number of degrees of freedom, such as one, two, three, four, five, six, seven, or more degrees-of-freedom. One or more motors (not shown) may drive movement of the first robotic arm 204 and/or the second robotic arm 208, such that the first robotic arm 204 and/or the second robotic arm 208 are capable of moving within a shared working environment, which may be a surgical environment. The first robotic arm 204 and/or the second robotic arm 208 may also comprise respective end effectors 224. The end effectors 224 may each connect to or mechanically couple with a surgical tool (e.g., a surgical drill, a surgical shaver, a surgical reamer, a surgical saw, etc.) or an implant device (e.g., a pedicle screw, a surgical rod, etc.) during the course of a surgery or surgical procedure. In some embodiments, the end effector 224 may enter or occupy a working volume to interact with patient anatomy.

The first robotic arm 204 and/or the second robotic arm 208 may move between one or more states 228A-228F. The states 228A-228F may be or comprise locations, sectors, volumes, positions, combinations thereof, and/or the like in 3D space that all links and tools of the first robotic arm 204 or the second robotic arm 208 may occupy. In some embodiments, the states 228A-228F may be volumes occupied by the first robotic arm 204 or the second robotic arm 208, such that when, for example, the first robotic arm 204 (or components thereof) occupies the volume (or portion of volume) of a first state, the navigation system 118 treats the robotic arm as being in the first state. Additionally or alternatively, the states 228A-228F may be defined by one or more sets of coordinates of the first robotic arm 204 or the second robotic arm 208. For example, a home state for the first robotic arm 204 may be defined as a state where the end effector 224 of the first robotic arm 204 is at an origin of a first coordinate system and where the remaining portions of the first robotic arm 204 (e.g., the segments 216 and the joints 220) are positioned such that the first robotic arm 204 is in a first pose. As such, any time the first robotic arm 204 is positioned in the first pose with the end effector 224 at the origin of the first coordinate system, the navigation system 118 may define the first robotic arm 204 as being in the home state.

The states 228A-228F may be determined preoperatively (e.g., before the surgery or surgical procedure begins) and/or intraoperatively (e.g., during the surgery or surgical procedure). In some embodiments, the states 228A-228F may be defined by the navigation system 118 and stored (e.g., in the database 130) for use during a surgery or surgical procedure. In some embodiments, the navigation system 118 may update the definition of the states 228A-228F intraoperatively. For example, the navigation system 118 may redefine a first state 228A after a new working volume has been determined. In some embodiments, the states 228A-228F may be associated with different areas in a surgical environment, such as a working volume state (e.g., a state where an end effector 224 and/or the one or more segments 216 of the first robotic arm 204 or the second robotic arm 208 is in a working volume), a tool cabinet state (e.g., a state where the first robotic arm 204 or the second robotic arm 208 is proximate a tool cabinet), a home state (e.g., a state where the first robotic arm 204 or the second robotic arm 208 is not in use, in a standby position, etc.), or the like. Additionally or alternatively, the states 228A-228F may be associated with different actions taken by the first robotic arm 204 or second robotic arm 208. For example, the states 228A-228F may comprise a right or left scanning state (e.g., a state where the first robotic arm 204 or the second robotic arm 208 moves an imaging device 112 along a respective right side length or a left side length of a patient to generate one or more images of patient anatomy), a right or left action state (e.g., a state where the first robotic arm 204 or the second robotic arm 208 moves a surgical tool into the working volume from a respective right or left side of the patient for the purposes of drilling, milling, sawing, cutting, etc. patient tissue), a right or left implant state (e.g., a state where the first robotic arm 204 or the second robotic arm 208 moves a surgical rod through one or more surgical screws on a respective right or left side of the patient), or the like. In such embodiments where the states 228A-228F represent actions, the states 228A-228F may be associated with one or more volumes or positions. For instance, while the home state may be defined as a single state with a fixed set of coordinates where the first robotic arm 204 or the second robotic arm 208 idles (or the volume that the first robotic arm 204 or the second robotic arm 208 occupies while idling), a scanning state may comprise a volume around the patient where the first robotic arm 204 or second robotic arm 208 moves to capture an image using, for example, the imaging device 112. In such embodiments, first robotic arm 204 or the second robotic arm 208 may be defined by the navigation system 118 as being in the scanning state any time the first robotic arm 204 or second robotic arm 208 occupies a part or all of the volume around the patient, as well as any time the first robotic arm 204 or the second robotic arm 208 are located at coordinates corresponding to a pose associated with the scanning state. In other words, as long as the first robotic arm 204 is occupying the volume or in a pose matching the performance of a task associated with the state, the first robotic arm 204 may be defined as occupying the state for the purposes of determining permissive and non-permissive states for the second robotic arm 208.

While the present disclosure depicts states 228A-228F, additional or alternative states may be defined. The number and type of states are in no way limited to what is discussed herein, and additional or alternative states of the first robotic arm 204 and the second robotic arm 208 are possible.

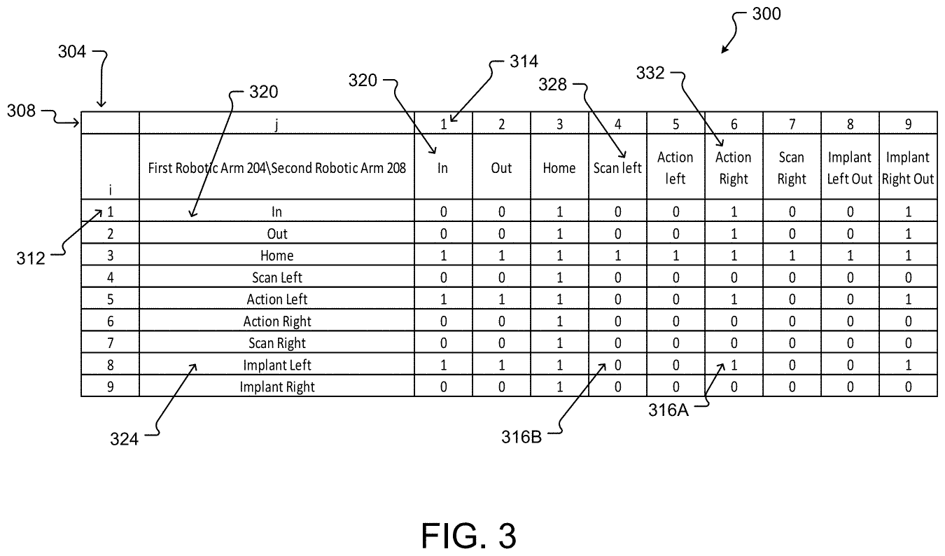

FIG. 3 depicts a combination state table 300 in accordance with at least one embodiment of the present disclosure. The combination state table 300 may be used as a reference for determining how the occupation of a first state by the first robotic arm 204 alters, changes, or impacts the types of states the second robotic arm 208 may occupy, and vice versa. The combination state table 300 may be maintained using a computing device 102 and/or one or more components thereof (e.g., a processor 104). In some embodiments, the combination state table 300 or data related thereto may be stored in a database 130, and extracted and processed by the processor 104 to determine acceptable and unacceptable states of the first robotic arm 204 based on the current state of the second robotic arm 208, and vice versa. In some embodiments, the combination state table 300 may be similar to or the same as the one or more state tables 138.

The combination state table 300 depicts a status identifier column 304 and a status identifier row 308. The status identifier column 304 may include values for a status identifier 312 associated with the first robotic arm 204, while the status identifier row 308 may include values for a status identifier 314 associated with the second robotic arm 208. The status identifier column 304 may show a range of all possible values the status identifier 312 may have, and the corresponding state of the first robotic arm 204 for each value. Similarly, the status identifier row 308 may show a range of all possible values the status identifier 314 may have, and corresponding states for the second robotic arm 208 for each value. For instance, as shown in FIG. 3, when the status identifier 312 has a value of five, the first robotic arm 204 is in an action left state, and when the status identifier 314 has a value of nine, the second robotic arm 208 is in an implant right out state.

The status identifiers 312, 314 may change in value based on the respective movement of the robotic arms 204, 208 between states. For example, the first robotic arm 204 may be in a first state with a status identifier 312 value of one. As the first robotic arm 204 moves to a second state, the status identifier 312 may be updated (e.g., from a value of one to a value of four), to reflect that the first robotic arm 204 is in the second state. In some embodiments, the computing device 102 or the navigation system 118 may update the status identifier 312 or the status identifier 314 as the respective first robotic arm 204 or second robotic arm 208 moves during the course of a surgery or surgical procedure, respectively. For instance, when the navigation system 118 causes the first robotic arm 204 to move from a home position to begin a right scanning action (e.g., providing an X-ray source for imaging of a patient's anatomy on the right side of the patient), the navigation system 118 may update or change the status identifier 312 from a first value (e.g., a value of three as depicted in the combination state table 300) to a second value (e.g., a value of seven as depicted in the combination state table 300). The navigation system 118 may then use the updated status identifier 312 along with the combination state table 300 to determine which states the second robotic arm 208 can and cannot move into.

The combination state table 300 may depict a plurality of states 320. Each state 320 may be similar to or the same as one of the states 228A-228F. In other words, a state 320 may represent a location or sector that a robotic arm may occupy. In some embodiments, the combination state table 300 may include a plurality of states for the first robotic arm 204 and the second robotic arm 208. In one embodiment, states 320 associated with the first robotic arm 204 may be shown in the second column of the combination state table 300, while states 320 associated with the second robotic arm 208 may be shown in the second row of the combination state table 300. In some embodiments, the combination state table 300 may contain a finite set or number of states. Stated differently, there may be a limited number of movements available for the first robotic arm 204 and/or the second robotic arm 208, with all possible movements and combinations of states for the first robotic arm 204 and the second robotic arm 208 listed in the combination state table 300. As a result, a matrix of values associated with each of the states may reflect all possible open or closed states for the second robotic arm 208 based on the current state of the first robotic arm 204, and vice versa.

The combination state table 300 may also show binary values 316A-316B for each of the states 320. The binary values 316A-316B may indicate whether the state is available for the first robotic arm 204 to enter based on the state of the second robotic arm 208, and vice versa. In some embodiments, the binary value 316A may be a value of one, which may indicate that the corresponding states 320 of the first robotic arm 204 and the second robotic arm 208 can be occupied at the same time, and the binary value 316B may be a value of zero, which may indicate that the corresponding states 320 of the first robotic arm 204 and the second robotic arm 208 cannot be occupied at the same time. For example, the combination state table 300 may include an implant left state 324 for the first robotic arm 204, which may be a state where the first robotic arm 204 implants a pedicle screw on the left side of the patient. The combination state table 300 may also include a scan left state 328 and an action right state 332 for the second robotic arm 208, which may be respective states where the second robotic arm 208 scans the left side of the patient to generate a medical image and moves a surgical tool along the right side of the patient. As shown in FIG. 3, when the first robotic arm 204 is in the implant left state 324 (and has a status identifier 312 value of eight), the second robotic arm 208 may be blocked from entering the scan left state 328, as shown by the binary value 316B of zero. In such embodiments, the navigation system 118 may prevent the second robotic arm 208 from entering the scan left state 328 (where the second robotic arm 208 has a status identifier 314 of four), to reduce the possibility of collision between the robotic arms, or for any other reason. In other words, the first robotic arm 204 being in the implant left state 324 and the second robotic arm 208 being in the scan left state 328 may position both robotic arms on the left side of the patient, increasing the probability of collision. However, the second robotic arm 208 may be able to enter the action right state 332 (corresponding to the second robotic arm 208 having a status identifier 314 of six) while the first robotic arm 204 is in the implant left state 324, as reflected by the binary value 316A of one in the combination state table 300.

The combination state table 300 may be or comprise a matrix of a predetermined set of binary values 316A-316B based on, for example, previous surgeries or surgical procedures. While binary values are shown in the combination state table 300, alternative values may be used. Additionally or alternatively, the navigation system 118 may begin by determining a first task for the first robotic arm 204, and then constructing the combination state table 300. For example, the navigation system 118 may determine that the first robotic arm 204 will perform a surgical implant in the working volume, and determine (e.g., using machine learning or artificial intelligent models) an appropriate set of movements of the first robotic arm 204 to accomplish the surgical implantation. The navigation system 118 may then determine one or more states that the second robotic arm 208 may occupy or not occupy based on the state of the first robotic arm 204 and fill in appropriate binary values in the combination state table 300. For example, during the surgical implant, the first robotic arm 204 may be inside the working volume, and the navigation system 118 may determine that the second robotic arm 208 should not pass into the working volume. Based on this information, the navigation system 118 may assign values of zero into the table for states associated with the second robotic arm 208 that include the second robotic arm 208 passing through or occupying the working volume.

In some embodiments, the combination state table 300 may be consulted by the computing device 102 and/or the navigation system 118 in planning and/or executing the motions of the first robotic arm 204 and the second robotic arm 208. For instance, the navigation system 118 may begin by determining a set of motions for the first robotic arm 204 associated with a first task (such as providing an imaging source to capture a preoperative image). As the first robotic arm 204 moves through the first set of motions and enters different states in performing the task, the status identifier 312 of the first robotic arm 204 may be updated (e.g., by the navigation system 118 based on the pose of the one or more segments 216 and/or a surgical tool of first robotic arm 204). For example, with reference to the combination state table 300, the first robotic arm 204 may begin with a status identifier 314 value of three, which may indicate that the first robotic arm 204 is in a home state (e.g., occupying a set of coordinates associated with the home state). The first robotic arm 204 may then be moved to a left side of the patient. Based on the resulting coordinates of the first robotic arm 204, the navigation system 118 (e.g., a processor 104 independently controlling the first robotic arm 204) may update the status identifier value from a value of three to a value of four, which indicates that the first robotic arm 204 is in a scan left state. Consequently, the change of the status identifier 314 from a value of three to a value of four may result in one or more states previously available to the second robotic arm 208 no longer being available. In the embodiment shown in FIG. 3, for example, the change in the status identifier 314 from a value of three to a value of four results in states associated with the status identifier 312 value of one, two, four, five, six, seven, eight, and nine being blocked or unavailable to the second robotic arm 208. In other words, an independent processor 104 or computing device 102 controlling the second robotic arm 208 may consult the combination state table 300 and determine that the second robotic arm 208 is no longer be able to move out of the home state while the first robotic arm 204 is in the scan left state.

In some embodiments, the navigation system 118 may cause the second robotic arm 208 to perform a second task (such as moving from a home state to a tool cabinet to collect a surgical rod for insertion into patient anatomy). As the first robotic arm 204 and the second robotic arm 208 perform respective tasks, the navigation system 118 may monitor the various states of the first robotic arm 204 and the second robotic arm 208 in the combination state table 300 and may update the movement of the first robotic arm 204 and/or the second robotic arm 208 to prevent collision of the two. For example, the first robotic arm 204, in moving the imaging source along the patient, may pass close to the tool cabinet. The navigation system 118 may respond by preventing the second robotic arm 208 from entering any state the causes the second robotic arm 208 to pass close to the tool cabinet. In some embodiments, the navigation system 118 may comprise two independent controllers, with each controller navigating a robotic arm independently of the other controller and robotic arm. As the controllers (e.g., using processors 104) navigate respective robotic arms, each controller may reference the combination state table 300 to ensure that the navigation of the robotic arm does not interfere or collide with the other robotic arm, and vice versa.

While the combination state table 300 depicts states with respect to the first robotic arm 204 and the second robotic arm 208, additional or alternative combination state tables may be used. Additionally, the number of robotic arms provided within the surgical environment and represented with the combination state table is in no way limited. For instance, three or more robotic arms may be present and used in conjunction with a multi-dimensional combination state table or multiple two-dimensional combination state tables. In such embodiments, the combination state table may comprise a plurality of state tables each comparing the states of two different robotic arms. Each state table may then be compared to the other state tables to determine open or blocked states for each robotic arm based on the states of the other robotic arms. In embodiments where multiple two-dimensional combination state tables are used, each two dimensional table may reflect a coordination between two specific robotic arms. With additional robotic arms, there may be an appropriate number of two dimensional tables for each pair or combination of robotic arms. For example, with four robotic arms, six state tables would be used to ensure that a state table exists for each pair or combination of robotic arms.

Alternatively, the combination state table may be generalized into a higher dimensional matrix containing binary values, with each dimension of the matrix containing binary values for each state of a respective robotic arm. For instance, with three robotic arms, a first dimension of the matrix may contain binary values for the first robotic arm, the second dimension of the matrix may contain binary values for the second robotic arm, and the third dimension of the matrix may contain binary values for the third robotic arm.

In some embodiments, the combination state table 300 may be rendered to a display (e.g., the user interface 110), to permit a user (e.g., a surgeon, a member of a surgical staff, etc.) to view the current states of the first robotic arm 204 and the second robotic arm 208. In some embodiments, the current state may be highlighted, such that the user can more clearly identify the current state of first robotic arm 204 and the second robotic arm 208. In some embodiments, as the status identifier 312 and status identifier 314 are updated, the rendering may be updated to highlight the new states.

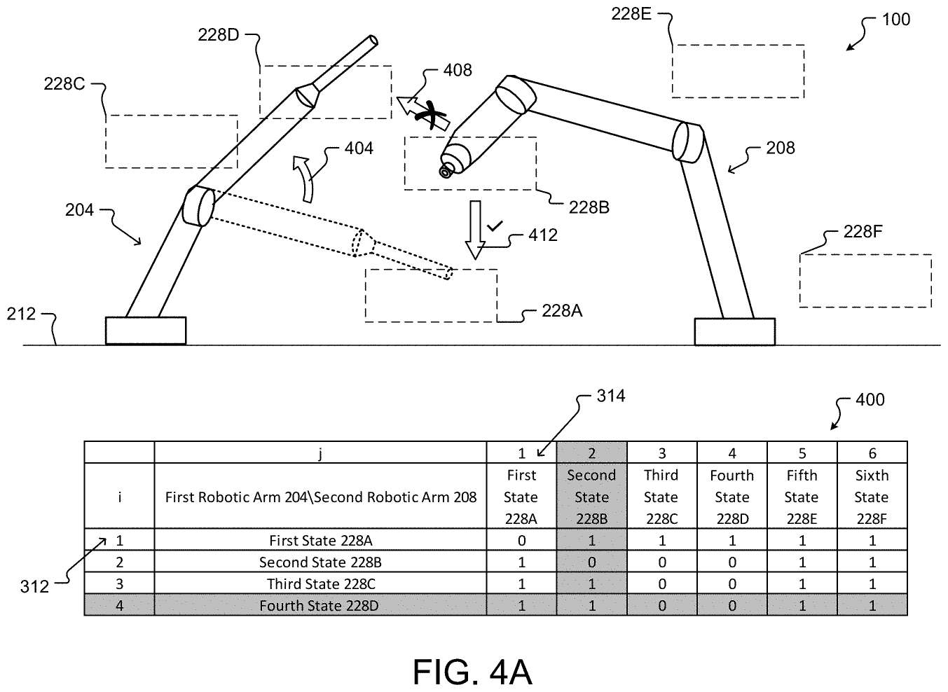

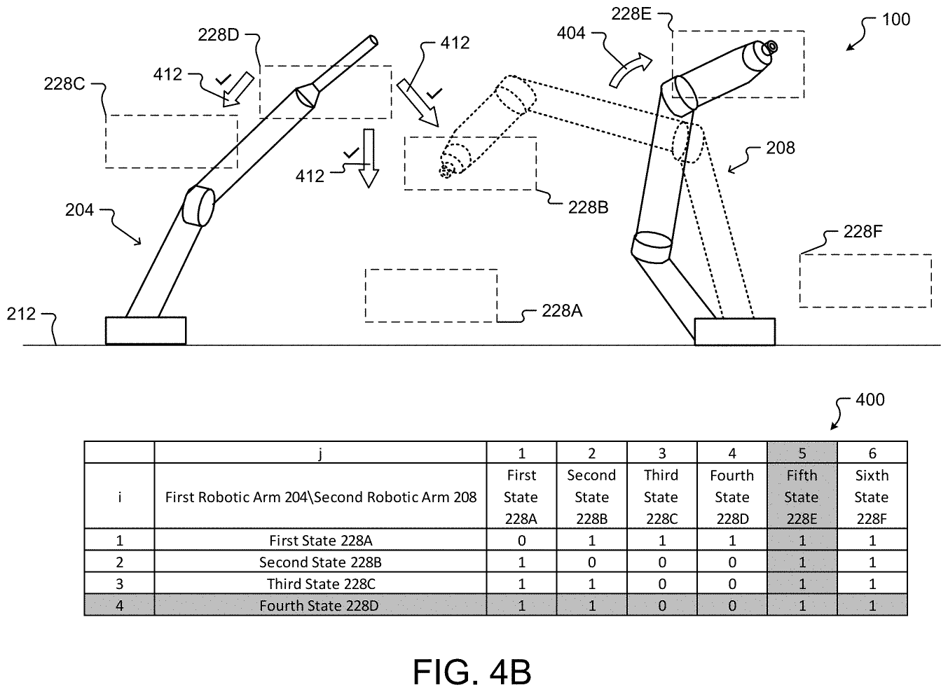

FIGS. 4A-4B depict hybrid motion of two robotic arms according to at least one embodiment of the present disclosure. The first robotic arm 204 and the second robotic arm 208 may be controlled to perform different tasks, such that the first robotic arm 204 and the second robotic arm 208 occupy various states 228A-228F throughout the course of the tasks. FIGS. 4A-4B also illustrate a combination state table 400. In some embodiments, the combination state table 400 may be similar to or the same as combination state table 300. The combination state table 400 may reflect various states 228A-228F that the first robotic arm 204 and the second robotic arm 208 may occupy while performing tasks during a surgery or surgical procedure. In some embodiments, one or more states may be unavailable to one of the robotic arms. For example, the first robotic arm 204 may be physically incapable of occupying one or more states (e.g., the first robotic arm 204 may be fixed to a surgical structure and is not long enough to move to the state) or may not be designed to occupy the one or more states (e.g., the first robotic arm 204 is a specialized robot for implanting surgical screws, and thus is not designed to occupy a state used to scan a patient to generate a surgical image). As shown in FIG. 4A, the first robotic arm 204 may be unable to occupy the fifth state 228E or the sixth state 228F. As such, any values associated with the first robotic arm 204 occupying the fifth state 228E or the sixth state 228F may be omitted from the combination state table 400.

The first robotic arm 204 may transition between states during the performance of one or more tasks. For example and as depicted in FIG. 4A, the first robotic arm 204 may begin in a first state 228A and the second robotic arm 208 may begin in the second state 228B, and the first robotic arm 204 may move from the first state 228A to a fourth state 228D, as illustrated with an arrow 404. The movement may be due to the first robotic arm 204 changing pose during the course of performing a tasks (e.g., moving to a tool cabinet to have a surgical tool attached to the end effector 224). As the first robotic arm 204 moves from the first state 228A to the fourth state 228D, the navigation system 118 may cause the status identifier 312 to be updated from a value associated with the first state 228A (e.g., a value of one) to a value associated with the fourth state 228D (e.g., a value of four).

The change in the status identifier 312 may enable the navigation system 118 to determine, based on the combination state table 400 and the values of the status identifiers 312, 314, a set of permissive states and a set of non-permissive states for the second robotic arm 208. In some embodiments, the combination state table 400 may be a matrix of binary values, with values of one reflective of permissive states and values of zero reflective of non-permissive states. While binary values are used herein for ease of discussion and understanding, it is possible for other values to be used in the state combination table 400, such that the permissive state may have a first known value associated therewith, while the non-permissive state may have a second known value associated therewith. As illustrated in FIG. 4A, the combination state table 400 indicates that, when the status identifier 312 for the first robotic arm 204 has a value of four, the second robotic arm 208 may occupy the first state 228A, the second state 228B, a fifth state 228E, and a sixth state 228F, and may not occupy the third state 228C or the fourth state 228D. Here, the first state 228A, second state 228B, fifth state 228E, and sixth state 228F constitute the set of permissive states that the second robotic arm 208 may move to occupy (as shown with a check mark arrow 412 from the second state 228B to the first state 228A). The third state 228C and the fourth state 228D constitute the set of non-permissive states that the second robotic arm 208 is not permitted to occupy (as shown with a crossed arrow 408 from the second state 228B to the fourth state 228D). Additionally or alternatively, the navigation system 118 may also determine sets of permissive states and sets of non-permissive states for the first robotic arm 204 based on the current state of the second robotic arm 208. Here, the combination state table 400 shows that, when the second robotic arm 208 is in the second state 228B, the set of permissive states for the first robotic arm 204 include the first state 228A, the third state 228C, and the fourth state 228D and the set of non-permissive states includes the second state 228B. In some embodiments, the set of non-permissive states may be states where the second robotic arm 208 has an increased probability of colliding with the first robotic arm 204 (and vice versa). As depicted in FIG. 4A, the positioning of the first robotic arm 204 in the fourth state 228D may obstruct the second robotic arm 208 from moving into the third state 228C, and there may be an increased risk of the second robotic arm 208 colliding with the first robotic arm 204 as the second robotic arm 208 attempts to move into the third state 228C.

In some embodiments, the navigation system 118 may use the sets of permissive and non-permissive states in controlling the second robotic arm 208. For instance, the navigation system 118 may navigate the second robotic arm 208 into any one of the first state 228A, the second state 228B, the fifth state 228E, or the sixth state 228F after the first robotic arm 204 is in the fourth state 228D but may block the second robotic arm 208 from entering the third state 228C or the fourth state 228D. In some embodiments, the navigation system 118 may provide real-time or near real-time (e.g., within 0.1 milliseconds (ms), within 0.2 ms, within 0.5 ms, within 1 ms, etc.) navigation updates to the second robotic arm 208 based on the change in state of the first robotic arm 204. For example, the navigation system 118 may plan to navigate the second robotic arm 208 into the fourth state 228D before the first robotic arm 204 moves to occupy the fourth state 228D. After the first robotic arm 204 has been moved to occupy the fourth state 228D, however, the navigation system 118 may update the navigation path of the second robotic arm 208 to prevent the second robotic arm 208 from passing into the fourth state 228D to reduce the probability of collision between the first robotic arm 204 and the second robotic arm 208.

In some embodiments, the task performed by the first robotic arm 204 may be fixed or otherwise pre-planned, such that the occupation of various states by the first robotic arm 204 is known. In other embodiments, the movement of the first robotic arm 204 may be adjusted based on the states occupied by the second robotic arm 208. For example, and as shown in FIG. 4B, while the first robotic arm 204 is in the fourth state 228D, the second robotic arm 208 may move out of the second state 228B and into the fifth state 228E (as shown with the arrow 404). The movement from the second state 228B to the fifth state 228E may cause the status identifier 314 associated with the second robotic arm 208 to update (e.g., from a value of two to a value of five). The update of the status identifier 314 may change the set of permissive states and set of non-permissive states for the first robotic arm 204. For example, while the second robotic arm 208 was in the second state 228B, the first robotic arm 204 was able to move to the first state 228A, the third state 228C, and the fourth state 228D, but not the second state 228B. However, once the second robotic arm 208 is in the fifth state 228E, the set of permissive states may include the second state 228B (as shown with the check mark arrows 412). In some embodiments, the first robotic arm 204 may be further controlled based on the set of permissive states and the set of non-permissive states. For instance, the first robotic arm 204 may be moved into one of the permissive states, or may be prevented from entering one of the non-permissive states (if any) when the second robotic arm 208 is in the fifth state 228E. Once the second robotic arm 208 has changed states (e.g., moved out of the fifth state 228E) the navigation system 118 may once again update the status identifier 314, determine new permissive and non-permissive states for the first robotic arm 204, and further control the first robotic arm 204 based on the updated set of permissive states and the updated set of non-permissive states.

In some embodiments, the combination state table 400 may be rendered to a display (e.g., the user interface 110) to provide a visual indicator to a user (e.g., a physician, a surgical staff member, etc.) of the current states of the first robotic arm 204 and the second robotic arm 208. In one embodiment, the combination state table 400 may be rendered to highlight the current states of the first robotic arm 204 and the second robotic arm 208 (e.g., different shading of the row and column of the matrix representing the current states). In some embodiments, the rendering may be updated as the first robotic arm 204 and/or the second robotic arm 208 change states to inform the user accordingly.

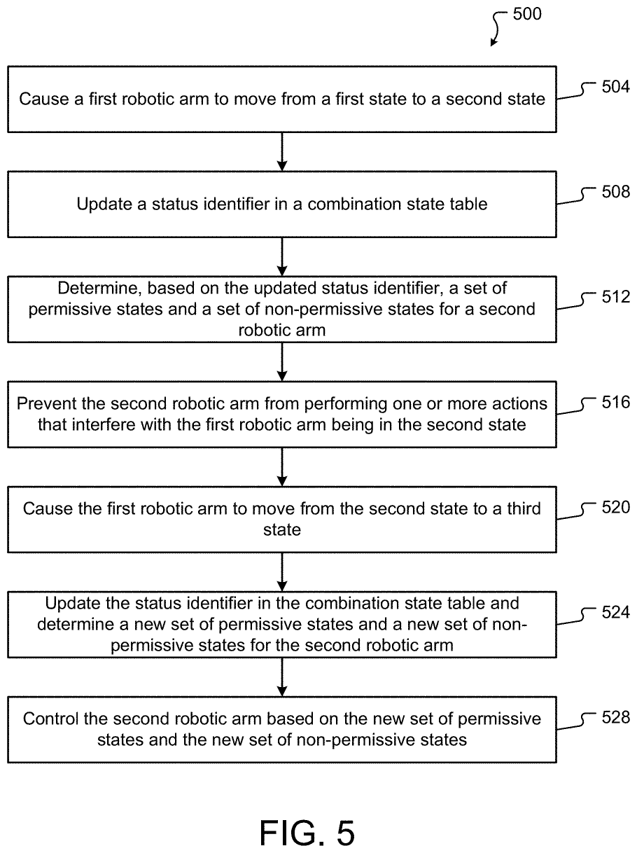

FIG. 5 depicts a method 500 that may be used, for example, to plan and execute one or more movements of two robotic arms.

The method 500 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 500. The at least one processor may perform the method 500 by executing elements stored in a memory such as the memory 106. The elements stored in memory and executed by the processor may cause the processor to execute one or more steps of a function as shown in method 500. One or more portions of a method 500 may be performed by the processor executing any of the contents of memory, such as an image processing 120, navigation 122, a transformation 124, and/or a registration 128.

The method 500 comprises causing a first robotic arm to move from a first state to a second state (step 504). The first robotic arm 204 may move from a first state (e.g., a first state 228A) to a second state (e.g., a second state 228B) based on a navigation path and/or during the course of performing a task during a surgery or surgical procedure. In some embodiments, the movement of the first robotic arm 204 may be predetermined based on a robotic planner (e.g., using a data model for navigation such as navigation 122) that uses mathematical tools (e.g., Inverse kinematics, gradient descent methods) to determine an optimized movement of the first robotic arm 204 in performing a task. In such embodiments, the states associated with the first robotic arm 204 may be known.

The movement of the first robotic arm 204 from the first state to the second state may occur as the first robotic arm 204 performs a task. For example, the first robotic arm 204 may be performing an implantation of a pedicle screw into a vertebra, and the transition from the first state to the second state may be or comprise the first robotic arm 204 moving from outside the working volume into the working volume to insert the implant. In such embodiments, the navigation system 118 may restrict the working volume such that only one robotic arm may occupy the working volume at a time.

The method 500 also comprises updating a status identifier in a combination state table (step 508). During or after the first robotic arm 204 has transitioned states, such as entering the working volume, the computing device 102 (or one or more components thereof) or the navigation system 118 may change the status identifier 312 associated with the first robotic arm 204 based on the state occupied in the combination state table 400. For example, the status identifier may initially be a value of one when the first robotic arm 204 is in the first state and may change to a value of two when the first robotic arm 204 moves into the second state. In some embodiments, the combination state table may be similar to or the same as the combination state table 300 and/or the combination state table 400.

The method 500 also comprises determining, based on the updated status identifier, a set of permissive states and a set of non-permissive state for a second robotic arm (step 512). After the status identifier 312 for the first robotic arm 204 has been updated, the navigation system 118 may determine a set of permissive states and a set of non-permissive states for the second robotic arm 208. For example, the set of permissive states may include the first state, a third state, and a fourth state (e.g., the first state 228A, the third state 228C, and the fourth state 228D), while the set of non-permissive states includes the second state (e.g., the second state 228B). In some embodiments, the navigation system 118 may also determine a set of permissive states and a set of non-permissive states for the first robotic arm 204 based on the status identifier of the second robotic arm 208 (and based on the combination state table).

The method 500 also comprises preventing the second robotic arm from performing one or more actions that interfere with the first robotic arm being in the second state (step 516). In some embodiments, the one or more actions may include moving the second robotic arm 208 into the second state 228B (e.g., into the working volume). Additionally or alternatively, the one or more actions may include moving the second robotic arm 208 into any states included in the set of non-permissive states for the second robotic arm 208. In some embodiments, the second robotic arm 208 may be prevented from performing the one or more actions as long as the first robotic arm 204 is in the second state. In other words, once the first robotic arm 204 exits the second state 228B (e.g., the first robotic arm 204 leaves the working volume), the second robotic arm 208 may be capable of performing the one or more actions (e.g., the second robotic arm 208 may then be able to enter the working volume). In some embodiments, the one or more actions may include navigating the second robotic arm 208 along a navigation path that passes the second robotic arm 208 into the non-permissive state. As an example, the second robotic arm 208 may be navigating along a first navigation path the causes the second robotic arm 208 to pass through the second state 228B (which may be a state associated with the working volume). In embodiments where the states are based on coordinates and poses of the robotic arms, the second state 228B may be associated with the second robotic arm 208 in a pose that places the end effector 224 of the second robotic arm 208 within the working volume. As the second robotic arm 208 moves along the first navigation path, the first robotic arm 204 may move into the second state 228B (e.g., the first robotic arm 204 enters the working volume). As a result, the updated status identifier 312 of the first robotic arm 204 may indicate that the second robotic arm 208 is no longer permitted in the second state 228B. As a result, the navigation system 118 may cause the second robotic arm 208 to change navigation from the first navigation to a second navigation path. The second navigation path may be designed to navigate the second robotic arm 208 without causing the second robotic arm 208 to pass through, intersect, or occupy the second state 228B.

The method 500 also comprises causing the first robotic arm to move from the second state to a third state (step 520). The movement from the second state to the third state may occur as the first robotic arm 204 performs a surgical task, such as implanting a screw into a vertebra. The third state may be the location where the first robotic arm 204 exits the working volume after having implanted the screw. In some embodiments, the third state may be a volume located along a right side of the patient (which may be where the first robotic arm 204 is positioned) that the first robotic arm 204 moves to occupy after exiting the second state (e.g., the working volume).

The method 500 also comprises updating the status identifier in the combination state table and determine a new set of permissive states and a new set of non-permissive states for the second robotic arm (step 524). In some embodiments, the step 524 may be similar to the steps 508 and 512 of the method 500 discussed above. For instance, the navigation system 118 may update the status identifier 312 associated with the first robotic arm 204, and may consult the combination state table to determine new permissive and non-permissive states for the second robotic arm 208. In one embodiment, the movement of the first robotic arm 204 to the right side of the patient may prevent the second robotic arm 208 from moving to the right side of the patient, as reflected by binary values of zero in the combination state table. Similarly, the movement of the first robotic arm 204 to the third state may open up the second state 228B (e.g., the working volume) to the second robotic arm 208 that was previously blocked by the first robotic arm 204, as reflected by a binary value of one in the combination state table.

The method 500 also comprises controlling the second robotic arm based on the new set of permissive states and the new set of non-permissive states (step 528). In some embodiments, the step 528 may be similar to or the same as the step 516 of the method 500 discussed above. In some embodiments, the controlling may include moving the second robotic arm 208 into any state in the new set of permissive states. For example, the second state 228B (e.g., the working volume) may have changed from a non-permissive state to a permissive state. As a result, the first navigation path (e.g., a path that moves the second robotic arm 208 through the second state 228B) may now be available to the navigation system 118. Similarly, states previously available to the second robotic arm 208 may now be blocked or otherwise unavailable. For instance, since the first robotic arm 204 has moved into the third state 228C (e.g., to the right of the patient), the third state 228C may be unavailable. Additionally or alternatively, states proximate to, related to, tied to, or dependent on the third state 228C may also be unavailable (e.g., states that require the second robotic arm 208 to be on the right side of the patient may be unavailable).

The present disclosure encompasses embodiments of the method 500 that comprise more or fewer steps than those described above, and/or one or more steps that are different than the steps described above.

As noted above, the present disclosure encompasses methods with fewer than all of the steps identified in FIG. 5 (and the corresponding description of the method 500), as well as methods that include additional steps beyond those identified in FIG. 5 (and the corresponding description of the method 500). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system, comprising:
a processor; and
a memory storing data thereon that, when processed by the processor, cause the processor to:
move a first robotic arm from a first state to a second state;
update, based on the moving of the first robotic arm from the first state to the second state, a first status identifier associated with the first robotic arm in a combination state table, the combination state table associated with the first robotic arm and a second robotic arm;
determine, based on the first status identifier and the combination state table, a set of permissive states and a set of non-permissive states for the second robotic arm; and
prevent the second robotic arm in a third state from performing one or more actions that interfere with the first robotic arm being in the second state.

2. The system of claim 1, wherein the combination state table comprises a matrix of binary values representative of a plurality of states, wherein the plurality of states comprises the first state, the second state, and the third state, wherein the combination state table represents a permissive state as a first known value, and wherein the combination state table represents a non-permissive state as a second known value.

3. The system of claim 2, wherein the set of non-permissive states includes the second state, and wherein the one or more actions includes the second robotic arm moving into the second state.

4. The system of claim 3, wherein the set of permissive states includes a fourth state, and wherein the data further cause the processor to:
move the second robotic arm from a third state into a fourth state;
update, based on the moving of the second robotic arm from the third state to the fourth state, a second status identifier associated with the second robotic arm in the combination state table; and
determine, based on the second status identifier and the combination state table, a permissive move and a non-permissive move for the first robotic arm.

5. The system of claim 4, wherein the data further cause the processor to:
prevent the first robotic arm from performing the non-permissive move.

6. The system of claim 2, wherein the combination state table includes a finite number of values.

7. The system of claim 6, wherein the plurality of states comprises a home state, a tool cabinet state, a scanning state, an action state, and an implant state.

8. The system of claim 1, wherein at least one of the first robotic arm or the second robotic arm comprises at least two degrees-of-freedom.

9. A method, comprising:
causing a first robotic arm to move from a first state to a second state;
updating, based on the first robotic arm moving from the first state to the second state, a first status identifier of the first robotic arm in a combination state table, the combination state table associated with both the first robotic arm and a second robotic arm;
determining, based on the first status identifier and the combination state table, at least one non-permissive state for the second robotic arm; and
preventing the second robotic arm in a third state from moving to the at least one non-permissive state while the first robotic arm is in the second state.

10. The method of claim 9, wherein the combination state table comprises a matrix of binary values representing a plurality of states, wherein the plurality of states comprise the first state, the second state, and the third state, wherein the combination state table represents a permissive state with a first known value, and wherein the combination state table represents a non-permissive state with a second known value.

11. The method of claim 10, wherein the at least one non-permissive state includes the second state.

12. The method of claim 11, further comprising:
causing the second robotic arm to move from the third state to a fourth state;
updating, based on the moving of the second robotic arm from the third state to the fourth state, a second status identifier associated with the second robotic arm in the combination state table;
determining, based on the second status identifier and the combination state table, a permissive move and a non-permissive move for the first robotic arm; and
preventing the first robotic arm from performing the non-permissive move.

13. The method of claim 12, wherein the second robotic arm begins moving from the third state to the fourth state along a first navigation path, and wherein preventing the second robotic arm from moving to the at least one non-permissive state includes:
updating, based on the at least one non-permissive state, the first navigation path to a second navigation path, wherein the first navigation path intersects the second state, and wherein the second navigation path avoids intersecting the second state.

14. The method of claim 13, further comprising:
permitting, upon the first robotic arm exiting the second state, the second robotic arm to move along the first navigation path.

15. The method of claim 10, wherein the plurality of states comprises a home state, a tool cabinet state, a scanning state, an action state, and an implant state.

16. The method of claim 9, wherein at least one of the first robotic arm or the second robotic arm comprises at least two degrees-of-freedom.

17. An apparatus, comprising:
a first robotic arm disposed in a first state of a plurality of states;
a second robotic arm disposed in a second state of the plurality of states;
a processor; and
a memory storing data thereon that, when processed by the processor, cause the processor to:
move, at a first time, the first robotic arm from the first state to a third state of the plurality of states;
update, based on the moving of the first robotic arm from the first state to the third state, a first status identifier associated with the first robotic arm in a combination state table, the combination state table associated with both the first robotic arm and the second robotic arm;
determine, based on the first status identifier and the combination state table, a set of non-permissive states for the second robotic arm; and
prevent, at a second time later than the first time, the second robotic arm from entering a non-permissive state of the set of non-permissive states.

18. The apparatus of claim 17, wherein preventing the second robotic arm from entering a non-permissive state includes:
diverting the second robotic arm from a first navigation path to a second navigation path, wherein the first navigation path intersects with the third state, and wherein the second navigation path avoids intersecting the third state.

19. The apparatus of claim 18, wherein the data further cause the processor to:
move, at a third time later than the second time, the first robotic arm from the third state to a fourth state of the plurality of states;
update, based on the move of the first robotic arm from the third state to the fourth state, the first status identifier associated with the first robotic arm in the combination state table; and
permit the second robotic arm to traverse the first navigation path.

20. The apparatus of claim 19, wherein the plurality of states include a home state, a tool cabinet state, a scanning state, an action state, and an implant state.
